(19) United States
(12) Patent Application Publication
(10) Pub. No.: US 2025/0206691 A1
(43) Pub. Date: Jun. 26, 2025

(54) PROCESS FOR THE RUTHENIUM-CATALYSED HYDROGENATION OF ALDEHYDE ACETALS

(71) Applicant: Evonik Oxeno GmbH & Co. KG, Marl (DE)

(72) Inventors: Thomas Schareina, Cammin (DE); Carolin Schneider, Monheim am Rhein (DE); Ralf Jackstell, Rostock (DE); Matthias Beller, Ostseebad Nienhagen (DE); Robert Franke, Marl (DE)

(73) Assignee: Evonik Oxeno GmbH & Co. KG, Marl (DE)

(21) Appl. No.: 18/980,557

(22) Filed: Dec. 13, 2024

(30) Foreign Application Priority Data
Dec. 22, 2023 (EP) 23219604.8

(51) Int. Cl. C07C 29/10; B01J 27/10

(52) U.S. Cl. CPC C07C 29/10; B01J 27/10; C07C 29/103

(57) ABSTRACT

Process for the ruthenium-catalysed hydrogenation of aldehyde acetals.

The present invention relates to a process for the ruthenium-catalysed hydrogenation of aldehyde acetals.

DE 42 20 939 A1 describes a process for preparing 2-aryl-ethanols:

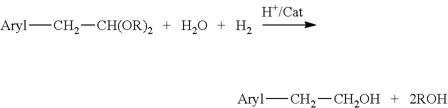

The reaction is effected in a sulfuric acid medium and is catalysed by an Ru/C catalyst, that is to say a supported Ru catalyst with carbon powder as support.

The present invention was based on the object of providing a process for the ruthenium-catalysed hydrogenation of aldehyde acetals that makes it possible to achieve a good yield.

This object is achieved by a process according to claim 1.

Process comprising the process steps of:
a) initially charging an aldehyde acetal of one of formulae (Ia) to (VIa):

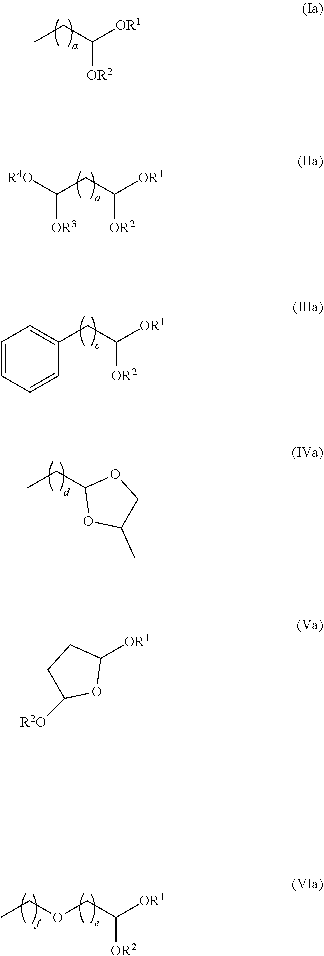

where a, c, d, f are an integer from 0 to 12 and b, e are an integer from 1 to 12,
and R¹, R², R³, R⁴, each independently, are (C₁-C₁₂)-alkyl;
b) adding an Ru compound capable of forming a complex and a ligand comprising a P atom, or an Ru-ligand complex, where the ligand of the complex comprises a P atom;
c) feeding in H₂;
d) heating the reaction mixture from a) to c), with conversion of the aldehyde acetal to a compound of formula (Ib) to (VIb):

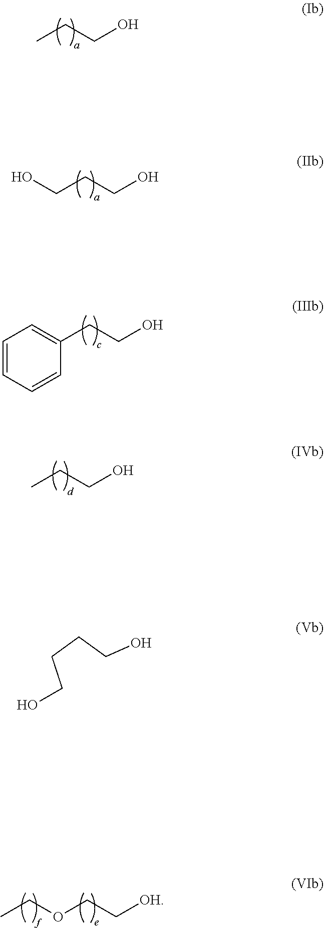

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

In one variant of the process, R¹, R² are the same radical.

In one variant of the process, R³, R⁴ are the same radical.

In one variant of the process, R¹, R², R³, R⁴ are the same radical.

In one variant of the process, R¹, R², R³, R⁴ are (C₁-C₄)-alkyl.

In one variant of the process, the Ru compound is selected from: RuCl₃×3H₂O, [Ru(cymene)Cl₂]₂, RuBr₃×3H₂O, RuI₃, Ru(PPh₃)₃Cl₂.

In one variant of the process, the ligand is a phosphine ligand or phosphite ligand.

In one variant of the process, the ligand is a phosphine ligand.

In one variant of the process, the ligand is selected from: PPh₃, 1,4-bis(diphenylphosphino)butane (dppb), 1,1′-ferrocenediylbis(diphenylphosphine) (dppf), bis[2-(diphenylphosphino)phenyl]ether (dpephos), 1,3-bis(diphenylphosphino)propane (dppp), 4,5-bis(diphenylphosphino)-9,9-dimethylxanthene (XantPhos).

In one variant of the process, H₂ is fed in with a pressure in the range from 0.5 MPa (5 bar) to 8 MPa (80 bar).

In one variant of the process, H₂ is fed in with a pressure in the range from 1 MPa (10 bar) to 6 MPa (60 bar).

In one variant of the process, the reaction mixture is heated to a temperature in the range from 30° C. to 100° C.

In one variant of the process, the reaction mixture is heated to a temperature in the range from 40° C. to 80° C.

In one variant of the process, the process comprises the additional process step c′):
c′) adding a solvent.

In one variant of the process, the solvent is selected from: 1,4-dioxane, tetrahydrofuran (THF), water.

The invention is to be elucidated in more detail hereinafter with reference to working examples.

### EXPERIMENTAL DESCRIPTION

### General (Autoclave)

An 8 ml vial was filled with the appropriate amounts of RuCl₃×3H₂O, PPh₃ and a magnetic stirrer. The vial was then sealed with a septum (PTFE-coated silicone rubber) and a phenolic resin cap. The vial was connected to the argon feed line via a needle. The vial was evacuated and filled with argon three times. An ether solvent (THF or 1,4-dioxane, stored under argon) and the appropriate amounts of water and substrate were injected into the vial using a syringe, with the result that a dark solution formed. The vial was placed into a stainless steel plate, with the needle still remaining in place in order to enable gas exchange in the autoclave. The plate was placed into an autoclave (300 ml) of the 4760 series from Parr Instruments under an argon atmosphere. After the autoclave had been purged three times with hydrogen, the hydrogen pressure was increased to 20 bar/40 bar at room temperature. The reaction was carried out by heating the autoclave in an aluminium block on a heating/stirring apparatus with magnetic stirring for 18 h at 60° C. (temperature of the aluminium block). At the end of the reaction time, the autoclave was cooled to room temperature and the pressure was cautiously released. Tetradecane (0.100 ml) was then injected as internal standard.

### A) 1,1-Dimethoxynonane to 1-nonanol

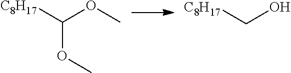

THE (1.5 ml), 0.135 ml (7.5 mmol) of water and 0.55 ml (0.47 g, 2.5 mmol) of 1,1-dimethoxynonane were added to 1.3 mg (0.2 mol %) of RuCl₃×3H₂O and 5.2 mg (0.8 mol %) of PPh₃. H₂ is injected to 20 bar and the reaction is carried out at 60° C. for 18 h. Yield (GC) quantitative.

### Variation of the Ligand

### 1,4-Bis(diphenylphosphino)butane (dppb)

Dioxane (1.5 ml, absolute), 0.36 ml (15 mmol) of water and 0.55 ml (0.47 g, 2.5 mmol) of 1,1-dimethoxynonane are added to 2.6 mg (0.4 mol %) of RuCl₃×3H₂O and 8.5 mg (0.04 mmol, 0.8 mol %) of 1,4-bis(diphenylphosphino)butane. H₂ is injected to 20 bar and the reaction is carried out at 60° C. for 18 h. Yield (GC) quantitative.

### 1,1′-Ferrocenediylbis(diphenylphosphine) (dppf)

Dioxane (1.5 ml, absolute), 0.36 ml (15 mmol) of water and 0.55 ml (0.47 g, 2.5 mmol) of 1,1-dimethoxynonane are added to 2.6 mg (0.4 mol %) of RuCl₃×3H₂O and 11.1 mg (0.04 mmol, 0.8 mol %) of 1,1′-ferrocenediylbis(diphenylphosphine). H₂ is injected to 20 bar and the reaction is carried out at 60° C. for 18 h. Yield (GC) quantitative.

### Bis[2-(diphenylphosphino)phenyl]ether (dpephos)

Dioxane (1.5 ml, absolute), 0.36 ml (15 mmol) of water and 0.55 ml (0.47 g, 2.5 mmol) of 1,1-dimethoxynonane are added to 2.6 mg (0.4 mol %) of RuCl₃×3H₂O and 10.8 mg (0.04 mmol, 0.8 mol %) of bis[2-(diphenylphosphino)phenyl]ether. H₂ is injected to 20 bar and the reaction is carried out at 60° C. for 18 h. Yield (GC) quantitative.

### 1,3-Bis(diphenylphosphino)propane (dppp)

Dioxane (1.5 ml, absolute), 0.36 ml (15 mmol) of water and 0.55 ml (0.47 g, 2.5 mmol) of 1,1-dimethoxynonane are added to 2.6 mg (0.4 mol %) of RuCl₃×3H₂O and 8.2 mg (0.04 mmol, 0.8 mol %) of 1,3-bis(diphenylphosphino)propane. H₂ is injected to 20 bar and the reaction is carried out at 60° C. for 18 h. Yield (GC) quantitative.

### 4,5-Bis(diphenylphosphino)-9,9-dimethylxanthene (XantPhos)

Dioxane (1.5 ml, absolute), 0.36 ml (15 mmol) of water and 0.55 ml (0.47 g, 2.5 mmol) of 1,1-dimethoxynonane are added to 2.6 mg (0.4 mol %) of RuCl₃×3H₂O and 11.6 mg (0.02 mmol, 0.8 mol %) of 4,5-bis(diphenylphosphino)-9,9-dimethylxanthene. H₂ is injected to 20 bar and the reaction is carried out at 60° C. for 18 h. Yield (GC) 59%.

### Variation of the Ru Compound

### [Ru(cymene)Cl₂]₂

Dioxane (1.5 ml, absolute), 0.36 ml (15 mmol) of water and 0.55 ml (0.47 g, 2.5 mmol) of 1,1-dimethoxynonane are added to 3.1 mg (0.4 mol % of ruthenium) of [Ru(cymene)Cl₂]₂ and 10.5 mg (0.04 mmol, 1.6 mol %) of PPh₃. H₂ is injected to 20 bar and the reaction is carried out at 60° C. for 18 h. Yield (GC) quantitative.

### RuBr₃×3H₂O

Dioxane (1.5 ml, absolute), 0.36 ml (15 mmol) of water and 0.55 ml (0.47 g, 2.5 mmol) of 1,1-dimethoxynonane are added to 3.9 mg (0.4 mol % of ruthenium) of RuBr₃×3H₂O and 10.5 mg (0.04 mmol, 1.6 mol %) of PPh₃. H₂ is injected to 20 bar and the reaction is carried out at 60° C. for 18 h. Yield (GC) 93%.

### RuI₃

Dioxane (1.5 ml, absolute), 0.36 ml (15 mmol) of water and 0.55 ml (0.47 g, 2.5 mmol) of 1,1-dimethoxynonane (0.47 g, 2.5 mmol) are added to 4.8 mg (0.4 mol % of ruthenium) of RuI₃ and 10.5 mg (0.04 mmol, 1.6 mol %) of PPh₃.

H₂ is injected to 20 bar and the reaction is carried out at 60° C. for 18 h. Yield (GC) 75%.

### Ru(PPh₃)₃Cl₂

Dioxane (1.5 ml, absolute), 0.36 ml (15 mmol) of water and 0.55 ml (0.47 g, 2.5 mmol) of 1,1-dimethoxynonane are added to 9.6 mg (0.4 mol % of ruthenium) of Ru(PPh₃)₃Cl₂ and 2.6 mg (0.01 mmol, 0.4 mol %) of PPh₃. H₂ is injected to 20 bar and the reaction is carried out at 60° C. for 18 h. Yield (GC) quantitative.

### Variation of the Aldehyde Acetal

### B) 1,1,6,6-Tetramethoxyhexane to hexane-1,6-diol

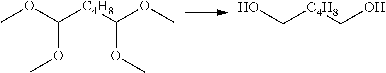

1,4-Dioxane (6 ml, absolute), 1.44 ml (80 mmol) of water and 2.06 g (10 mmol) of 1,1,6,6-tetramethoxyhexane, 10.5 mg (0.4 mol %) of RuCl₃×3H₂O and 42 mg (1.6 mol %) of PPh₃ are added to a vial. H₂ is injected to 40 bar and the reaction is carried out at 60° C. for 18 h. The GC yield is 81%.

### C) 1,1,6,6-Tetrabutoxyhexane to hexane-1,6-diol

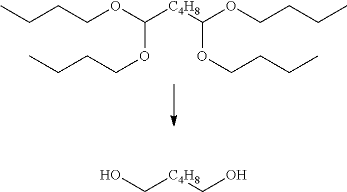

1.5 ml of THF, 0.99 g (2.64 mmol) of 1,1,6,6-tetrabutoxyhexane and 0.72 ml of H₂O, 2.6 mg (0.38 mol %) of RuCl₃×3H₂O and 10.5 mg (1.52 mol %) of PPh₃ are added to a vial. H₂ is injected to 20 bar and the reaction is carried out at 60° C. for 18 h. The GC yield is 90%.

### D) 1,1,4,4-Tetramethoxybutane to butane-1,4-diol

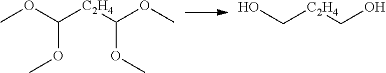

1.5 ml of dioxane, 0.54 ml of water and 0.46 g (2.56 mmol) of 1,1,4,4-tetramethoxybutane, 2.6 mg (0.39 mol %) of RuCl₃×3H₂O and 10.5 mg (1.56 mol %) of PPh₃ are added to a vial. H₂ is injected to 20 bar and the reaction is carried out at 60° C. for 18 h. The NMR yield is 66%.

### E) Benzaldehyde Dimethyl Acetal to Benzyl Alcohol

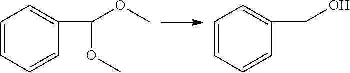

1.5 ml of THF, 0.135 g of H₂O and 0.42 g (2.72 mmol) of benzaldehyde dimethyl acetal, 1.3 mg (0.18 mol %) of RuCl₃×3H₂O and 5.2 mg (0.73 mol %) of PPh₃ are added to a vial. H₂ is injected to 20 bar and the reaction is carried out at 60° C. for 18 h. The GC yield is >99%.

### F) Phenylacetaldehyde Dimethyl Acetal to 2-Phenylethanol

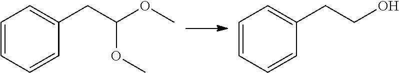

1.5 ml of dioxane, 0.36 g of H₂O and 0.42 g (2.5 mmol) of phenylacetaldehyde dimethyl acetal, 2.6 mg (0.4 mol %) of RuCl₃×3H₂O and 10.5 mg (1.6 mol %) of PPh₃ are added to a vial. H₂ is injected to 20 bar and the reaction is carried out at 60° C. for 18 h. The GC yield is >99%.

### G) 4-Methyl-2-octyl-1,3-dioxolane to 1-nonanol

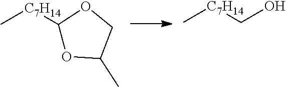

1,4-Dioxane (1.5 ml, absolute), 0.135 ml (7.5 mmol) of water and 0.5 ml of 4-methyl-2-octyl-1,3-dioxolane (0.47 g, 2.3 mmol), 2.6 mg (0.4 mol %) of RuCl₃×3H₂O and 10.5 mg (1.6 mol %) of PPh₃ are added to a vial. H₂ is injected to 40 bar and the reaction is carried out at 60° C. for 18 h. The GC yield is 89%.

### H) 2,5-Dimethoxytetrahydrofuran (Cis/Trans Mixture) to Butane-1,4-Diol

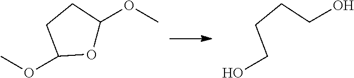

1.5 ml of dioxane, 0.18 ml of water and 0.33 g (2.5 mmol) of 2,5-dimethoxytetrahydrofuran (cis/trans mixture), 2.6 mg of RuCl₃×3H₂O and 10.5 mg (1.6 mol %) of PPh₃ are added to a vial. H₂ is injected to 20 bar and the reaction is carried out at 60° C. for 18 h. The GC yield is 84%.

### I) 3-Ethoxypropionaldehyde diethyl acetal to 3-ethoxypropanol

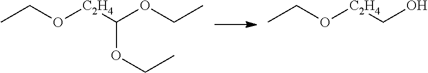

1.5 ml of dioxane, 0.27 g of H₂O and 0.451 g (2.56 mmol) of ethoxypropionaldehyde diethyl acetal, 2.6 mg (0.39 mol %) of RuCl₃×3H₂O and 10.5 mg (1.56 mol %) of PPh₃ are added to a vial. H₂ is injected to 20 bar and the reaction is carried out at 60° C. for 18 h. The GC yield is >99%.

### Variation of the Catalyst System

### J) Phenylacetaldehyde dimethyl acetal to 2-phenylethanol

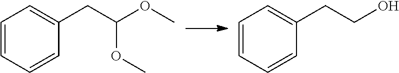

The solid catalyst was weighed out in 8 ml vials and all liquids were added with the syringe. The substrate was added last here.

The following catalyst systems were tested:
Cat 1: 0.4 mol % of RuCl₃×3 H₂O, 1.6 mol % of PPh₃, 0.36 ml of water, 1.5 ml of dioxane
Cat 2: Ru/C 5% (Strem) 44-4065 LOT #:21539500, 50% water content, calculated for 0.67 mol % of metal, 0.17 ml of 0.1 M H₂SO₄ (aq), 1.34 ml of water, 1.5 ml of methanol
Cat 3: Ru/C 5% (Johnson-Matthey) Type 622, LOT KS0004, 0.17 ml of 0.1 M H₂SO₄ (aq), 1.34 ml of water, 1.5 ml of methanol

Diglyme was added, after the reaction, as GC standard.

### Reaction Conditions:

2.5 mmol of substrate, 20 bar of H₂, 60° C., 5 h.

The experimental results are listed in the following table:

| Catalyst | Yield |
|---|---|
| Cat 1* | >98% |
| Cat 2 | 52% |
| Cat 3 | 56% |

*catalyst system according to the invention

As the experimental results show, the object is achieved by the process according to the invention.

1. Process comprising the process steps of:
a) initially charging an aldehyde acetal of one of formulae (Ia) to (VIa): 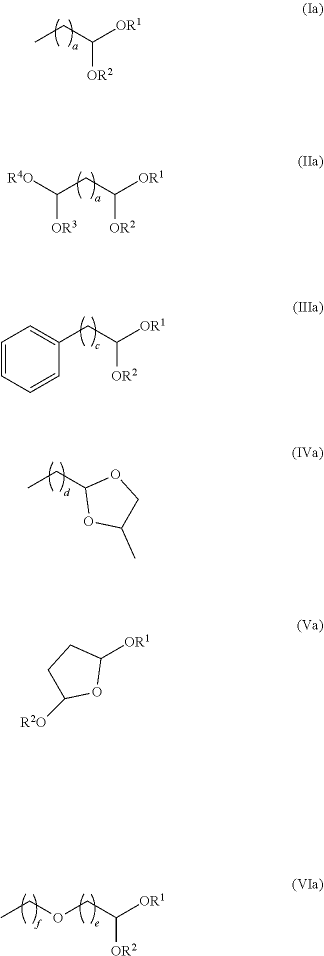
where a, c, d, f are an integer from 0 to 12 and b, e are an integer from 1 to 12
and R¹, R², R³, R⁴, each independently, are (C₁-C₁₂)-alkyl;
b) adding an Ru compound capable of forming a complex and a ligand comprising a P atom, or an Ru-ligand complex, where the ligand of the complex comprises a P atom;
c) feeding in H₂;
d) heating the reaction mixture from a) to c), with conversion of the aldehyde acetal to a compound of formula (Tb) to (VIb):

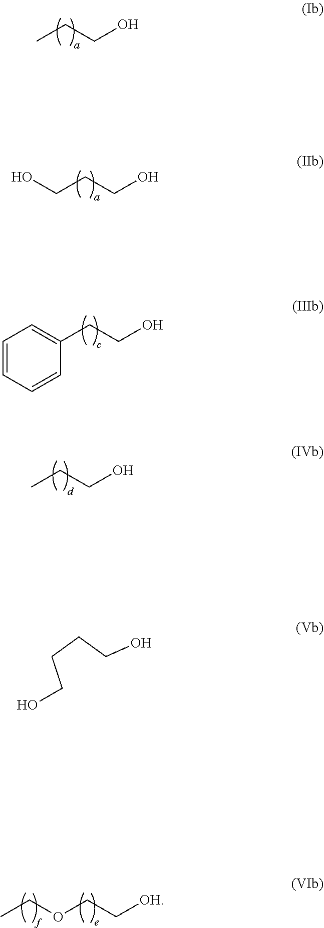

2. Process according to claim 1,
wherein R¹, R² are the same radical.

3. Process according to claim 1,
wherein R³, R⁴ are the same radical.

4. Process according to claim 1,
wherein R¹, R², R³, R⁴ are (C₁-C₄)-alkyl.

5. Process according to claim 1,
wherein the Ru compound is selected from: RuCl₃×3H₂O, [Ru(cymene)Cl₂]₂, RuBr₃×3H₂O, RuI₃, Ru(PPh₃)₃Cl₂.

6. Process according to claim 1,
wherein the ligand is a phosphine ligand.

7. Process according to claim 1,
wherein the ligand is selected from: PPh₃, 1,4-bis(diphenylphosphino)butane (dppb), 1,1′-ferrocenediylbis(diphenylphosphine) (dppf), bis[2-(diphenylphosphino)phenyl]ether (dpephos), 1,3-bis(diphenylphosphino)propane (dppp), 4,5-bis(diphenylphosphino)-9,9-dimethylxanthene (XantPhos).

8. Process according to claim 1,
wherein H₂ is fed in with a pressure in the range from 0.5 MPa (5 bar) to 8 MPa (80 bar).

9. Process according to claim 1,
wherein the reaction mixture is heated to a temperature in the range from 30° C. to 100° C.

10. Process according to claim 1,
comprising the additional process step c′):
c′) adding a solvent.

11. Process according to claim 10,
wherein the solvent is selected from: 1,4-dioxane, tetrahydrofuran (THF), water.
